(19) United States
(12) Patent Application Publication
(10) Pub. No.: US 2011/0055977 A1
(43) Pub. Date: Mar. 3, 2011

(54) Enhancement of Reproductive Heat Tolerance in Plants

(76) Inventor: John J. Burke, Lubbock, TX (US)

(21) Appl. No.: 12/549,895

(22) Filed: Aug. 28, 2009

(51) Int. Cl. A01H 5/00; C12N 15/82; C12N 5/10; A01H 5/10

(52) U.S. Cl. 800/289; 800/298; 800/314; 800/320.1; 800/320.3; 800/312; 800/320; 435/419

(57) ABSTRACT

The reproductive heat tolerance of plants may be enhanced by transformation with chimeric construct comprising a nucleic acid coding sequence encoding a heat shock protein operatively linked to a promoter which is effective for expression in mature pollen of the plant. Although mature pollen of plants do not normally express heat shock proteins, plants transformed with this construct express and accumulate the heat shock protein even in pollen which is mature. The mature pollen of the transformed plants exhibits significantly increased tolerance to elevated temperature stress.

### BACKGROUND OF THE INVENTION

1. Field of the Invention

The invention is drawn to the use of thermotolerance enhancing proteins to improve the heat tolerance of pollen.

2. Description of the Prior Art

Comparison of average crop yields with reported record yields has shown that the major crops grown in the U.S. exhibit annual average yields three- to seven-fold lower than record yields because of unfavorable environments. Analysis of yields from corn, wheat, soybeans, sorghum, oats, barley, potatoes, and sugar beets revealed that the average yield represented only 22% of the mean record yield (Boyer, 1982, Science, 218:443-448). Crops with economically valuable reproductive structures showed the greatest discrepancy between average and record yields. Evaluation of crop losses between 1948 and 1989 by the Federal Crop Insurance Corporation showed that on average, 69% of insurance indemnities could be attributed to drought and excess heat in barley, canning beans, corn, forages, oat, peanut, rye, safflower, soybean, and wheat.

Numerous organisms, including plants, elicit heat shock responses upon exposure to temperature extremes. These responses include the production of the well-known heat shock proteins which strengthen the capacity of the organism to survive at these temperature extremes. The transformation of a variety of organisms with genes coding for different heat shock proteins has been investigated as a means for enhancing the expression of the heat shock proteins and enhancing this survival. For example, Lindquist (U.S. Pat. No. 5,827,685) disclosed transformation of plant with a construct of the hsp104 gene under the control of the ³⁵S cauliflower mosaic virus promoter to facilitate the survival of the transformed plants at high temperatures.

However, despite these and other advances, the need remains for improved techniques for enhancing plant reproduction and health under temperature extremes.

### SUMMARY OF THE INVENTION

I have now discovered that the reproductive heat tolerance of plants may be enhanced through the use of a DNA construct comprising a nucleic acid coding sequence encoding a heat shock protein operatively linked to a promoter which is effective for expression in mature pollen of the plant. Although mature pollen of plants do not normally express heat shock proteins, I have found that the mature pollen of plants transformed with this construct do express and accumulate the encoded heat shock protein therein. The mature pollen of the transformed plants exhibit significantly increased tolerance to elevated temperature stress.

The transgenic plants of this invention which comprise mature pollen exhibiting increased heat tolerance may be produced from any plant, plant tissue or plant cell which is capable of regeneration, by transformation with the construct comprising a nucleic acid coding sequence encoding a heat shock protein operatively linked to a promoter effective for expression in mature pollen of the plant. Transformed plants, plant tissue or plant cells comprising the construct are selected, and the transgenic plant is regenerated therefrom.

In accordance with this discovery, it is an object of this invention to provide a process for producing plants with increased reproductive heat tolerance.

It is another object of this invention to provide plants which produce mature pollen expressing heat shock protein.

Yet another object of this invention is to provide transformed plants which produce mature pollen that exhibit significantly increased tolerance to elevated temperature stress and enhanced germination in comparison to an non-transformed control plants.

Other objects and advantages of this invention will become readily apparent from the ensuing description.

### BRIEF DESCRIPTION OF THE DRAWINGS

FIG. 1 shows a diagram of pE1801-ocs/mas ‘superpromoter’-HSP101 plasmid of Example 1. PAg7 is the Transcription termination and poly-Adenylation signal sequence from Octopine Ti-Plasmid T-DNA (gene for transcript #7) [Velten and Schell, 1985, Selection-expression vectors for use in genetic transformation of higher plants, Nucleic Acids Res., 13:6981-6998). NptII is the Neomycin phosphotransferase II coding region (Fraley et al., 1986]. Pnos is the Nopaline synthase promoter from Nopaline Ti-Plasmid T-DNA [Koncz et al., 1983, The opine synthase genes carried by Ti plasmids contain all signals necessary for expression in plants, EMBO J., 2(9):1597-603]. Aocs X 3 is the Octopine synthase enhancer element (3 copies) from Octopine T-Plasmid T-DNA [Bouchez et al., 1989, EMBO J., The ocs-element is a component of the promoters of several T-DNA and plant viral genes, 8(13):4197-204]. AmasPmas is the Manopine synthase promoter from Octopine Ti-Plasmid T-DNA [Velten et al., 1984, Isolation of a dual plant promoter fragment from the Ti plasmid of Agrobacterium tumefaciens, EMBO J., 3:2723-2730]. HSP101 is the Heat shock protein (101 kdalton molecular weight) from A. thaliana [Queitsch et al., 2000 The Plant Cell, 12:479-492]. Ags-ter is the Transcription termination and poly-Adenylation signal sequence from Octopine Ti-Plasmid T-DNA (Agropine synthase gene) [Bandyopadhyay et al., 1989, Regulatory elements within the agropine synthase promoter of T-DNA, J. Biol. Chem., 264(32):19399-406].

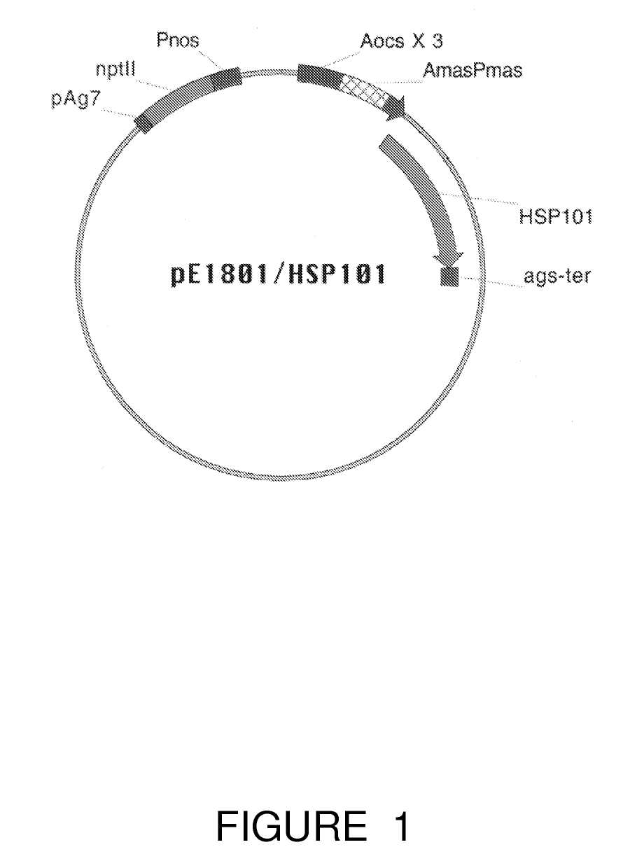

FIG. 2 is a graph of tobacco pollen tube lengths following either control or heat treatment in Example 1.

FIG. 3 shows a graph of control and transgenic cotton pollen germination at 23° C. and 39° C. in Example 2.

FIG. 4 shows a graph of high and low temperatures during field studies in Maricopa, AZ in Example 3. Temperatures over 100° F. were common throughout the flowering and boll development period.

FIG. 5 shows a graph of the number of bolls per plant from the Maricopa, AZ field study in Example 3.

### DEFINITIONS

The following terms are employed herein:

Cloning. The selection and propagation of (a) genetic material from a single individual, (b) a vector containing one gene or gene fragment, or (c) a single organism containing one such gene or gene fragment.

Cloning Vector. A plasmid, virus, retrovirus, bacteriophage, cosmid, artificial chromosome (bacterial or yeast), or nucleic acid sequence which is able to replicate in a host cell, characterized by one or a small number of restriction endonuclease recognition sites at which the sequence may be cut in a predetermined fashion, and which may contain an optional marker suitable for use in the identification of transformed cells, e.g., tetracycline resistance or ampicillin resistance. A cloning vector may or may not possess the features necessary for it to operate as an expression vector.

Codon. A DNA sequence of three nucleotides (a triplet) which codes (through mRNA) for an amino acid, a translational start signal, or a translational termination signal. For example, the nucleotide triplets TTA, TTG, CTT, CTC, CTA, and CTG encode for the amino acid leucine, while TAG, TAA, and TGA are translational stop signals, and ATG is a translational start signal.

DNA Coding Sequence. A DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, procaryotic sequences and cDNA from eukaryotic mRNA. A polyadenylation signal and transcription termination sequence will usually be located 3′ to the coding sequence.

DNA Construct. Artificially constructed (i.e., non-naturally occurring) DNA molecules useful for introducing DNA into host cells, including chimeric genes, expression cassettes, and vectors.

DNA Sequence. A linear series of nucleotides connected one to the other by phosphodiester bonds between the 3′ and 5′ carbons of adjacent pentoses.

Expression. The process undergone by a structural gene to produce a polypeptide. Expression requires transcription of DNA, post-transcriptional modification of the initial RNA transcript, and translation of RNA.

Expression Cassette. A chimeric nucleic acid construct, typically generated recombinantly or synthetically, which comprises a series of specified nucleic acid elements that permit transcription of a particular nucleic acid in a host cell. In an exemplary embodiment, an expression cassette comprises a heterologous nucleic acid to be transcribed, operably linked to a promoter. Typically, an expression cassette is part of an expression vector.

Expression Control Sequence. Expression control sequences are DNA sequences involved in any way in the control of transcription or translation and must include a promoter. Suitable expression control sequences and methods of making and using them are well known in the art.

Expression Vector. A nucleic acid which comprises an expression cassette and which is capable of replicating in a selected host cell or organism. An expression vector may be a plasmid, virus, retrovirus, bacteriophage, cosmid, artificial chromosome (bacterial or yeast), or nucleic acid sequence which is able to replicate in a host cell, characterized by a restriction endonuclease recognition site at which the sequence may be cut in a predetermined fashion for the insertion of a heterologous DNA sequence. An expression vector may include the promoter positioned upstream of the site at which the sequence is cut for the insertion of the heterologous DNA sequence, the recognition site being selected so that the promoter will be operatively associated with the heterologous DNA sequence. A heterologous DNA sequence is “operatively associated” with the promoter in a cell when RNA polymerase which binds the promoter sequence transcribes the coding sequence into mRNA which is then in turn translated into the protein encoded by the coding sequence.

Fusion Protein. A protein produced when two heterologous genes or fragments thereof coding for two different proteins not found fused together in nature are fused together in an expression vector. For the fusion protein to correspond to the separate proteins, the separate DNA sequences must be fused together in correct translational reading frame.

Gene. A segment of DNA which encodes a specific protein or polypeptide, or RNA.

Genome. The entire DNA of an organism. It includes, among other things, the structural genes encoding for the polypeptides of the substance, as well as operator, promoter and ribosome binding and interaction sequences.

Heterologous DNA. A DNA sequence inserted within or connected to another DNA sequence which codes for polypeptides not coded for in nature by the DNA sequence to which it is joined. Allelic variations or naturally occurring mutational events do not give rise to a heterologous DNA sequence as defined herein.

Hybridization. The pairing together or annealing of single stranded regions of nucleic acids to form double-stranded molecules.

Nucleotide. A monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. The base characterizes the nucleotide. The four DNA bases are adenine (“A”), guanine (“G”), cytosine (“C”), and thymine (“T”). The four RNA bases are A, G, C, and uracil (“U”).

Operably Linked, Encodes or Associated. Operably linked, operably encodes or operably associated each refer to the functional linkage between a promoter and nucleic acid sequence, wherein the promoter initiates transcription of RNA corresponding to the DNA sequence. A heterologous DNA sequence is “operatively associated” with the promoter in a cell when RNA polymerase which binds the promoter sequence transcribes the coding sequence into mRNA which is then in turn translated into the protein encoded by the coding sequence.

Phage or Bacteriophage. Bacterial virus many of which include DNA sequences encapsidated in a protein envelope or coat (“capsid”). In a unicellular organism a phage may be introduced by a process called transfection.

Plant. Plant refers to a unicellular organism or a multicellular differentiated organism capable of photosynthesis, including algae, angiosperms (monocots and dicots), gymnosperms (ginko, cycads, gnetophytes, and conifers), bryophytes, ferns and fern allies. Plant parts are parts of multicellular differentiated plants and include seeds, pollen, embryos, flowers, fruits, shoots, leaves, roots, stems, explants, etc.

Plant Cell. Plant cell refers to the structural and physiological unit of multicellular plants. Thus, the term plant cell refers to any cell that is a plant or is part of, or derived from, a plant. Some examples of cells encompassed by the present invention include differentiated cells that are part of a living plant, differentiated cells in culture, undifferentiated cells in culture, and the cells of undifferentiated tissue such as callus or tumors.

Plasmid. A non-chromosomal double-stranded DNA sequence comprising an intact “replicon” such that the plasmid is replicated in a host cell. When the plasmid is placed within a unicellular organism, the characteristics of that organism may be changed or transformed as a result of the DNA of the plasmid. A cell transformed by a plasmid is called a “transformant.”

Polypeptide. A linear series of amino acids connected one to the other by peptide bonds between the alpha-amino and carboxy groups of adjacent amino acids.

Promoter. A DNA sequence within a larger DNA sequence defining a site to which RNA polymerase may bind and initiate transcription. A promoter may include optional distal enhancer or repressor elements. The promoter may be either homologous, i.e., occurring naturally to direct the expression of the desired nucleic acid, or heterologous, i.e., occurring naturally to direct the expression of a nucleic acid derived from a gene other than the desired nucleic acid. A promoter may be constitutive or inducible. A constitutive promoter is a promoter that is active under most environmental and developmental conditions. An inducible promoter is a promoter that is active under environmental or developmental regulation, e.g., upregulation in response to wounding of plant tissues. Promoters may be derived in their entirety from a native gene, may comprise a segment or fragment of a native gene, or may be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. It is further understood that the same promoter may be differentially expressed in different tissues and/or differentially expressed under different conditions.

Reading Frame. The grouping of codons during translation of mRNA into amino acid sequences. During translation the proper reading frame must be maintained. For example, the DNA sequence may be translated via mRNA into three reading frames, each of which affords a different amino acid sequence.

Recombinant DNA Molecule. A hybrid DNA sequence comprising at least two DNA sequences, the first sequence not normally being found together in nature with the second.

Ribosomal Binding Site. A nucleotide sequence of mRNA, coded for by a DNA sequence, to which ribosomes bind so that translation may be initiated. A ribosomal binding site is required for efficient translation to occur. The DNA sequence coding for a ribosomal binding site is positioned on a larger DNA sequence downstream of a promoter and upstream from a translational start sequence.

Replicon. Any genetic element (e.g., plasmid, chromosome, virus) that functions as an autonomous unit of DNA replication in vivo, i.e., capable of replication under its own control.

Start Codon. Also called the initiation codon, is the first mRNA triplet to be translated during protein or peptide synthesis and immediately precedes the structural gene being translated. The start codon is usually AUG, but may sometimes also be GUG.

Structural Gene. A DNA sequence which encodes through its template or messenger RNA (mRNA) a sequence of amino acids characteristic of a specific polypeptide.

Transform. To change in a heritable manner the characteristics of a host cell in response to DNA foreign to that cell. An exogenous DNA has been introduced inside the cell wall or protoplast. Exogenous DNA may or may not be integrated (covalently linked) to chromosomal DNA making up the genome of the cell. In prokaryotes and yeast, for example, the exogenous DNA may be maintained on an episomal element such as a plasmid. With respect to eucaryotic cells, a stably transformed cell is one in which the exogenous DNA has been integrated into a chromosome so that it is inherited by daughter cells through chromosome replication. This stability is demonstrated by the ability of the eucaryotic cell to establish cell lines or clones comprised of a population of daughter cells containing the exogenous DNA.

Transcription. The process of producing mRNA from a structural gene.

Transgenic plant. A plant comprising at least one heterologous nucleic acid sequence that was introduced into the plant, at some point in its lineage, by genetic engineering techniques. Typically, a transgenic plant is a plant that is transformed with an expression vector. It is understood that a transgenic plant encompasses a plant that is the progeny or descendant of a plant that is transformed with an expression vector and which progeny or descendant retains or comprises the expression vector. Thus, the term “transgenic plant” refers to plants which are the direct result of transformation with a heterologous nucleic acid or transgene, and the progeny and descendants of transformed plants which comprise the introduced heterologous nucleic acid or transgene.

Translation. The process of producing a polypeptide from mRNA.

### DETAILED DESCRIPTION OF THE INVENTION

Crop yield data suggests that plants have high productivity potentials, but are operating well below their genetic potential because of the heat sensitivity of the reproductive structures. Indeed, Dupuis and Dumas (1990, Plant Physiol., 94:665-670) described that in maize, mature pollen is sensitive to heat stress, and is responsible for the failure of fertilization under heat shock conditions. The authors further disclosed that in contrast with female reproductive tissues, the mature pollen of maize does not express heat shock proteins even under heat shock conditions. The invention described herein provides a solution to the natural sensitivity of the male reproductive structures, providing a method for expressing heat shock proteins in mature pollen which are not normally expressed therein, but which proteins are reported to play a crucial role in vegetative thermotolerance (Queitsch et al., 2000, The Plant Cell, 12:479-492).

In accordance with this invention, heat shock proteins may be expressed in mature pollen by use of DNA constructs wherein the nucleic acid coding sequence encoding a heat shock protein is operatively linked to a promoter which is active (i.e., functional or effective for expression) in mature pollen of the plant of interest. Thus, the selection of the appropriate promoter is critical. In contrast with the normal plants, plants which are transformed with this construct produce mature pollen which express and accumulate the heat shock protein therein. Moreover, the encoded heat shock proteins are expressed at a level sufficient in the mature pollen of these transformed plants such that the mature pollen exhibits significantly increased tolerance, and particularly increased germination rates, upon exposure to high temperature stress, than pollen of non-transformed control plants.

A variety of heat shock proteins (HSP) are suitable for use herein, and may be of eukaryotic (animal, plant, or protist) or prokaryotic origin, and may be from the same or different species as the host plant of interest. Moreover, the proteins may be cognate (i.e., expressed in normal cells in the absence of temperature stress) or inducible (i.e., produced in normal cells in response to temperature stress). However, use of inducible heat shock proteins is preferred. As will be discussed in greater detail hereinbelow, the selection of a cognate or inducible protein is distinct from the selection of the promoter(s) in the construct. Without being limited thereto, suitable heat shock proteins which may be used herein include those in the families HSP 100 or 110 (this family has been referred to by different authors as HSP 100 or HSP 110, but each refer to those HSPs having a molecular weight range between approximately 100 and 110 kDa), HSP 90 (HSPs ranging in size between approximately 80 to 94 kDa), HSP 70, HSP 60, and low molecular weight (LMW) HSPs (recognized in the art as those having a molecular weight between 15 and 30 kDa). Heat shock proteins of the HSP 70 family, and particularly the HSP 100 family, are preferred. Numerous heat shock proteins within these families and their corresponding nucleic acid coding sequences have been isolated and described, for example, by Schoffl et al. (1998, Plant Physiol., 117:1135-1141), Schoffl et al. (Molecular Responses to Heat Stress. IN: Molecular Responses to Cold, Drought, Heat, and Salt Stress in Higher Plants, R. G. Landes publisher, 1999, pp. 81-88), Vierling (1991, Annu. Rev. Plant Physiology Plant Mol. Biol., 42:579-620), Nover (1997, Cellular and Molecular Life Sciences, 53:80-103), Lindquist (U.S. Pat. No. 5,827,685), and Zimmerman et al. (U.S. Pat. No. 5,922,929), and any one of these HSPs may be suitable for use herein. By way of example, preferred heat shock proteins (and the nucleic acid sequences which encode them) for use herein include Arabidopsis thaliana heat shock protein 101 (Queitsch et al., 2000, The Plant Cell, 12:479-492), and carrot HSP 17.7 (Zimmerman et al., U.S. Pat. No. 5,922,929). The contents of each of the publications and patents referred to hereinabove are incorporated by reference herein.

As noted above, the promoter selected must be active in mature pollen, but because many promoters are inactive in pollen, the selection of the promoter is critical. Promoters suitable for use herein should provide a level of expression of the heat shock protein in the mature pollen of the resultant transgenic plant, such that this mature pollen will exhibit significantly increased tolerance to elevated temperature stress, in comparison to the pollen of non-transformed or wild-type control plants. As used herein, an elevated temperature stress is defined as a prolonged exposure of a growing target plant to temperatures which are substantially greater than those which are optimal for growth (i.e., yield) of the same control (non-transformed) plant. The actual temperature which constitutes an elevated temperature stress will of course vary with the particular crop of interest and the variety thereof, soil conditions, and geography, and may be readily determined by the skilled practitioner. A variety of promoters are effective for expression in mature pollen and are suitable for use herein. In a preferred embodiment, the constitutive ocs/mas superpromoter (Ni et al., 1995, The Plant Journal 7(4):661-676; and Lee et al., 2007, Plant Physiology 145:1294-1300) is used in the process of this invention. Other pollen active promoters which are suitable for use herein include, but are not limited to, the PMT1 promoter as described by Garrido et al. [2006, Promoter activity of a putative pollen monosaccharide transporter in Petunia hybrida and characterization of a transposon insertion mutant, Protoplasma, 228(1-3):3-11]; the Lupme3 promoter as described by Lacoux et al. [2003, Activity of a flax pectin methylesterase promoter in transgenic tobacco pollen, Journal of Plant Physiology, 160(8):977-979]; the SbgLR promoter as described by Lang et al. [2007, Functional characterization of the pollen-specific SBgLR promoter from potato (Solanum tuberosum L.), Planta, 227(2):387-396]; an alfalfa promoter as described by Wu et al. [1998, A comparison of the promoter regions of three pollen-specific genes in alfalfa, Sexual Plant Reproduction, 11(3):181-182]; the maize ZM13 promoter as described by Hamilton et al. [1998, A monocot pollen-specific promoter contains separable pollen-specific and quantitative elements, Plant Molecular Biology, 38:663-669; 1992, Dissection of a pollen-specific promoter from maize by transient transformation assays, Plant Molecular Biology: an International Journal on Molecular Biology, Biochemistry and Genetic Engineering, 18:211-218; and 2000, Comparison of transient and stable expression by a pollen-specific promoter: the transformation results do not always agree, Sexual Plant Reproduction, 12:292-295]; the Sta 44G(2) promoter as described by Hong et al. [1997, The promoter of a Brassica napus polygalacturonase gene directs pollen expression of beta-glucuronidase in transgenic Brassica plants, Plant Cell Reports, 16:363-367]; the PsEND1 promoter as described by Piston et al. [2008, The pea PsEND1 promoter drives the expression of GUS in transgenic wheat at the binucleate microspore stage and during pollen tube development, Molecular Breeding, 21(3):401-405]; the g10 promoter as described by Rogers et al. [2001, Functional analysis of cis-regulatory elements within the promoter of the tobacco late pollen gene g10, Plant Molecular Biology, 45:577-585]; the Bra r 1 promoter as described by Okada et al. [2000, Expression of Bra r 1 gene in transgenic tobacco and Bra r 1 promoter activity in pollen of various plant species, Plant and Cell Physiology, 41:757-766]; the LAT52 promoter as described by Gerola et al. [2000, Regulation of LAT52 promoter activity during pollen tube growth through the pistil of Nicotiana alata. Sexual Plant Reproduction, 12:347-352]; the Lhca3.St.1 promoter as described by Conner et al. [1999, Gametophytic expression of GUS activity controlled by the potato Lhca3.St.1 promoter in tobacco pollen, Journal of Experimental Botany, 50:1471-1479]; the G9 promoter as described by John and Petersen [1994, Cotton (Gossypium hirsutum L.) pollen-specific polygalacturonase mRNA: tissue and temporal specificity of its promoter in transgenic tobacco, Plant Molecular Biology, 26:1989-1993]; the NTP303 promoter as described by Weterings et al. [1995, Functional dissection of the promoter of the pollen-specific gene NTP303 reveals a novel pollen-specific, and conserved cis-regulatory element, Plant Journal : for Cell and Molecular Biology, 8:55-63]; and the DEFH125 promoter as described by Lauri et al. [2006, The pollen-specific DEFH125 promoter from Antirrhinum is bound in vivo by the MADS-box proteins DEFICIENS and GLOBOSA, Planta, 224(1):61-71]. The contents of each of these publications referred to hereinabove are incorporated by reference herein.

Various methods may be used to produce the DNA construct, expression cassette or vector comprising the pollen active promoter and heat shock protein sequences for transformation of the desired plant or its tissue or cells. The skilled artisan is well aware of the genetic elements that must be present on an expression construct/vector in order to successfully transform, select and propagate the expression construct in host cells. Techniques for manipulation of nucleic acids encoding promoter and protein sequences such as subcloning nucleic acid sequences into expression vectors, labeling probes, DNA hybridization, and the like are described generally in Sambrook et al., [Molecular Cloning—A Laboratory Manual (2nd Ed.), Vol. 1-3, Cold Spring Harbor Laboratory, Cold Spring Harbor, N.Y., 1989] and Kriegler [Gene Transfer and Expression: A Laboratory Manual, 1990].

DNA constructs comprising the pollen active promoter operably linked to the heat shock protein DNA sequence can be inserted into a variety of vectors. Typically, the vector chosen is an expression vector that is useful in the transformation of plants and/or plant cells. Moreover, the expression constructs will typically comprise restriction endonuclease sites to facilitate vector construction and ensure that the promoter is upstream of and in-frame with the heat shock protein sequence. Exemplary restriction endonuclease recognition sites include, but are not limited to recognition site for the restriction endonucleases NotI, AatII, SacII, PmeI HindIII, PstI, EcoRI, and BamHI.

The expression vector may be a plasmid, virus, cosmid, artificial chromosome, nucleic acid fragment, or the like. Such vectors can be constructed by the use of recombinant DNA techniques well known to those of skill in the art. The expression vector comprising the promoter sequence may then be transfected/transformed into the target host cells. Successfully transformed cells are then selected based on the presence of a suitable marker gene as disclosed below.

A variety of vectors may be used to create the expression constructs comprising the pollen active promoter and operably linked heat shock protein sequences. Numerous recombinant vectors are known and available to those of skill in the art and are suitable for use herein for use in the stable transfection of plant cells or for the establishment of transgenic plants (see e.g., Weissbach and Weissbach, (1989) Methods for Plant Molecular Biology, Academic Press; Gelvin et al., (1990) Plant Molecular Biology Manual; Genetic Engineering of plants, an Agricultural Perspective, A. Cashmore, Ed.; Plenum: NY, 1983; pp 29 38; Coruzzi, G. et al., The Journal of Biological Chemistry, 258:1399 (1983); and Dunsmuir, P. et al., Journal of Molecular and Applied Genetics, 2:285 (1983). The choice of the vector is influenced by the method that will be used to transform host plants, and appropriate vectors are readily chosen by one of skill in the art.

Typically, the plant transformation vectors will include the pollen active promoter sequences operably linked to the heat shock protein gene (or cDNA sequence) in the sense orientation, and a selectable marker. Such plant transformation vectors may also include a transcription initiation start site, a ribosome binding site, an RNA processing signal, a transcription termination site, and/or a polyadenylation signal. The plant transformation vectors may also include additional regulatory sequences from the 3′-untranslated region of plant genes, e.g., a 3′ terminator region to increase mRNA stability of the mRNA, such as the PI-II terminator region of potato or the octopine or nopaline synthase (NOS) 3′ terminator regions. The expression constructs may further comprise an enhancer sequence such that the expression of the heterologous protein may be enhanced. As is known in the art, enhancers are typically found 5′ to the start of transcription, they can often be inserted in the forward or reverse orientation, either 5′ or 3′ to the coding sequence. Expression constructs prepared as disclosed herein will typically also include a sequence that acts as a signal to terminate transcription and allow for the poly-adenylation of the mRNA produced by heat shock protein coding sequences operably linked to the promoter. Termination sequences are typically located in the 3′ flanking sequence of a coding sequence, which will typically comprise the proper signals for transcription termination and polyadenylation. Thus, in one embodiment, termination sequences are ligated into the expression vector 3′ of the heat shock protein coding sequences to provide polyadenylation and termination of the mRNA. Terminator sequences and methods for their identification and isolation are known to those of skill in the art, see e.g., Albrechtsen, B. et al. (1991) Nucleic Acids Res. April 25; 19(8): 1845-1852, and WO/2006/013072. The transcription termination sequences comprising the expression constructs, may also be associated with known genes from the host organism. Yet other DNA sequences encoding additional functions may also be present in the vector, as is known in the art. For instance, in the case of Agrobacterium transformations, T-DNA sequences will also be included for subsequent transfer to plant chromosomes

As noted above, plant transformation vectors typically include a selectable and/or screenable marker gene to allow for the ready identification of transformants. As is known in the art, marker genes are genes that impart a distinct phenotype to cells expressing the marker gene, such that transformed cells can be distinguished and/or selected from cells that do not have the marker (and thus have not incorporated the vector). Exemplary selectable marker genes include, but are not limited to, those encoding antibiotic resistance (e.g. resistance to hygromycin, kanamycin, bleomycin, G418, streptomycin or spectinomycin) and herbicide resistance genes (e.g., phosphinothricin acetyltransferase). In this embodiment, the marker genes encode a selectable marker which one can “select” for by chemical means, e.g., through the use of a selective agent (e.g., a herbicide, antibiotic, or the like). Alternatively, the marker genes may encode a screenable marker which is identified through observation or testing, e.g., by “screening” Exemplary screenable markers include e.g., green fluorescent protein.

A variety of selectable marker genes are known to the art and are suitable for use herein. Some exemplary selectable markers are disclosed in e.g., Potrykus et al. (1985, Mol. Gen. Genet., 199:183-188); Stalker et al. (1988, Science, 242:419 422); Thillet et al. (1988, J. Biol. Chem., 263:12500 12508); Thompson et al. (1987, EMBO J. 6:2519-2523); Deblock et al. (1987, EMBO J. 6:2513-2518); U.S. Pat. No. 5,646,024; U.S. Pat. No. 5,561,236; U.S. Patent application Publication 20030097687; and Boutsalis and Powles (1995, Weed Research 35: 149-155). Screenable markers suitable for use herein include, but are not limited to, a β-glucuronidase (GUS) or uidA gene, (see e.g., U.S. Pat. No. 5,268,463, U.S. Pat. No. 5,432,081 and U.S. Pat. No. 5,599,670); a β-gene (see e.g., Sutcliffe, 1978, Proc. Natl. Acad. Sci. USA, 75:3737-3741); β-galactosidase; and luciferase (lux) gene [see e.g., Ow et al., 1986, Science, 234:856-859; Sheen et al., 1995, Plant J., 8(5):777-784; and WO 97/41228]. Other suitable selectable or screenable marker genes also include genes which encode a “secretable marker” whose secretion can be detected as a means of identifying or selecting for transformed cells. Such secretable markers include, but are not limited to, secretable antigens that can be identified by antibody interaction (e.g., small, diffusible proteins detectable for example by ELISA); secretable enzymes which can be detected by their catalytic activity, such as small active enzymes detectable in extracellular solution (e.g., a-amylase, β-lactamase or phosphinothricin acetyltransferase); and proteins that are inserted or trapped in the cell wall (e.g., proteins that include a leader sequence such as that found in the expression unit of extensin or tobacco PR-S).

The DNA constructs containing the pollen active promoter operably linked to the heat shock protein DNA sequence can be used to transform plants, plant tissue or plant cells and and thereby generate transgenic plants which produce mature pollen exhibiting increased tolerance to heat. Plants which may be transformed in accordance with this invention may be dicotyledonous or monocotyledonous species, and include, but are not limited to sorghum (Sorghum vulgare), alfalfa (Medicago saliva), sunflower (Helianthus annus), soybean (Glycine max), tobacco (Nicotiana tabacum), potato (Solanum tuberosum), peanuts (Arachis hypogaea), cotton (Gossypium hirsutum), sweet potato (Ipomoea batatus), cassava (Manihot esculenta), wheat (Triticum spp), rice (Oryza sativa), barley (Hordeum vulgare), oats (Avena sativa), maize (Zea mays), rye (Secale cereale), onion (Allium spp), pineapple (Ananas comosus), citrus trees (Citrus spp.), cocoa (Theobroma cacao), tea (Camellia sinensis), banana (Musa spp.), avocado (Persea americana), fig (Ficus casica), papaya (Carica papaya), almond (Prunus amygdalus), sugar beets (Beta vulgaris), apple (Malus pumila), blackberry (Rubus), strawberry (Fragaria), walnut (Juglans regia), grape (Vitis vinifera), apricot (Prunus armeniaca), cherry (Prunus), peach (Prunus persica), plum (Prunus domestica), pear (Pyrus communis), watermelon (Citrullus vulgaris), tomatoes; (Solanum lycopersicum), lettuce (e.g., Lactuea sativa), carrots (Caucuis carota), cauliflower (Brassica oleracea), celery (apium graveolens), eggplant (Solanum melongena), asparagus (Asparagus officinalis), ochra (Abelmoschus esculentus), green beans (Phaseolus vulgaris), lima beans (Phaseolus limensis), peas (Lathyrus spp.), members of the genus Cucurbita, e.g., Hubbard squash (C. Hubbard), Butternut squash (C. moschtata), Zucchini (C. pepo), Crookneck squash (C. crookneck), C. argyrosperma, C. argyrosperma ssp sororia, C. digitata, C. ecuadorensis, C. foetidissima, C. lundelliana, and C. martinezii, and members of the genus Cucumis such as cucumber (Cucumis sativus), cantaloupe (C. cantalupensis), and musk melon (C. melo). Ornamental plants e.g., azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherima), and chrysanthemum, and laboratory plants, e.g., Arabidopsis. Of these, cotton, maize, wheat, soybeans, sorghum, oats, and barley are preferred.

Transformation of plant, plant tissue or plant cell with the DNA construct comprising the nucleic acid sequence encoding a heat shock protein operatively linked to the pollen active promoter may be effected using a variety of known techniques. Techniques for the transformation and regeneration of monocotyledonous and dicotyledonous plant cells are well known in the art, see e.g., Weising et al., 1988, Ann. Rev. Genet. 22:421-477; U.S. Pat. No. 5,679,558; Agrobacterium Protocols Kevan M. A. Gartland ed. (1995) Humana Press Inc.; and Wang, M., et al., 1998, Acta Hort. (ISHS) 461:401-408. A variety of techniques are suitable for use herein, and include, but are not limited to, electroporation, microinjection, microprojectile bombardment, also known as particle acceleration or biolistic bombardment, viral-mediated transformation, and Agrobacterium-mediated transformation. The choice of the preferred method for use herein will vary with the type of plant to be transformed, the particular application and/or the desired result, and may be readily determined by the skilled practitioner. Detailed descriptions of transformation/transfection methods are available disclosed, for example, as follows: direct uptake of foreign DNA constructs (see e.g., EP 295959); techniques of electroporation [see e.g., Fromm et al., 1986, Nature (London) 319:791]; high-velocity ballistic bombardment with metal particles coated with the nucleic acid constructs [see e.g., Kline et al., 1987, Nature (London) 327:70, and U.S. Pat. No. 4,945,050]; methods to transform foreign genes into commercially important crops, such as rapeseed [see De Block et al., 1989, Plant Physiol. 91:694-701], sunflower [Everett et al., 1987, Bio/Technology 5:1201], soybean [McCabe et al., 1988, Bio/Technology 6:923; Hinchee et al., 1988, Bio/Technology 6:915; Chee et al., 1989, Plant Physiol. 91:1212 1218; Christou et al., 1989, Proc. Natl. Acad. Sci. USA 86:7500 7504; EP 301749], rice [Hiei et al., 1994, Plant J. 6:271 282], corn [Gordon-Kamm et al., 1990, Plant Cell 2:603-618; Fromm et al., 1990, Biotechnology 8:833 839], and Hevea (Yeang et al., In, Engineering Crop Plants for Industrial End Uses. Shewry, P. R., Napier, J. A., David, P. J., Eds. Portland: London, 1998, pp 55-64). Other suitable, known methods are disclosed in e.g., U.S. Pat. Nos. 5,597,945; 5,589,615; 5,750,871; 5,268,526; 5,262,316; and 5,569,831. In a preferred embodiment the transformation is effected using Agrobacterium-meditated transformation.

Agrobacterium tumefaciens-meditated transformation techniques are well described in the scientific literature. See, e.g., Horsch et al. Science, 1984, 233:496-498, and Fraley et al., 1983, Proc. Natl. Acad. Sci. USA 80:4803. Typically, a plant cell, an explant, a meristem or a seed is infected with Agrobacterium tumefaciens transformed with the expression vector/construct which comprises the pollen active promoter and heat shock protein DNA sequence. Under appropriate conditions known in the art, the transformed plant cells are grown to form shoots, roots, and develop further into plants. The nucleic acid segments can be introduced into appropriate plant cells, for example, by means of the Ti plasmid of Agrobacterium tumefaciens. The Ti plasmid is transmitted to plant cells upon infection by Agrobacterium tumefaciens, and is stably integrated into the plant genome (Horsch et al., 1984, Inheritance of Functional Foreign Genes in Plants, Science, 233:496-498; and Fraley et al., 1983, Proc. Nat'l. Acad. Sci. U.S.A. 80:4803).

After transformation of the plant, plant cell or tissue, those plant cells or plants transformed with the selected vector such that the construct is integrated therein can be cultivated in a culture medium under conditions effective to grow the plant or its cell or tissue. Successful transformants may be differentiated and selected from non-transformed plants or cells using a phenotypic marker. As described above, these phenotypic markers include, but are not limited to, antibiotic resistance, herbicide resistance or visual observation.

Transformed plant cells which are derived by any of the above transformation techniques can be cultured to regenerate a whole plant which possesses the desired transformed phenotype of increased tolerance of pollen to elevated temperatures. Plant regeneration techniques are well known in the art. For example, plant regeneration from cultured protoplasts is described in Evans et al., Protoplasts Isolation and Culture, Handbook of Plant Cell Culture, pp. 124-176, MacMillan Publishing Company, New York, 1983; and Binding, Regeneration of Plants, Plant Protoplasts, pp. 21-73, CRC Press, Boca Raton, 1985, all of which are incorporated herein by reference. Regeneration can also be obtained from plant callus, explants, organs, or parts thereof. Such regeneration techniques are described generally in Klee et al. 1987, Ann. Rev. of Plant Phys. 38:467-486, the contents of which is also incorporated by reference herein.

The skilled artisan will recognize that different independent transformation events will result in different levels and patterns of expression (Jones et al., 1985, EMBO J., 4:2411 2418; and De Almeida et al., 1989, Mol. Gen. Genetics, 218:78 86), and thus that multiple events may need to be screened in order to obtain lines displaying the desired expression level of the heat shock protein. Exemplary methods for screening transformation events may be accomplished e.g., by Southern analysis of DNA blots (Southern, 1975, J. Mol. Biol., 98: 503), Northern analysis of mRNA expression [Kroczek, 1993, Chromatogr. Biomed. Appl., 618(1-2): 133-145] and/or Western analysis of protein expression. Expression of the heterologous heat shock protein DNA can also be detected by measurement of the specific RNA transcription product. This can be done, for example, by RNAse protection or Northern blot procedures, or by antibody analyses. In another exemplary embodiment, protein expression is quantitated and/or detected in different plant tissues using a reporter gene, e.g., GUS.

In any event, in the preferred embodiment, transformed plants are screened for the desired increase in tolerance of the mature pollen to elevated temperature stress. Mature pollen of transformed plants which express the heat shock protein at a sufficient level therein, will exhibit significantly increased tolerance to elevated temperature stress (heat), in comparison to the pollen of non-transformed or wild-type control plants. As described in the Examples hereinbelow, increased tolerance to elevated temperature stress may be demonstrated, for example, by significantly enhanced germination (i.e., increased pollen viability), increased fruiting (i.e., increased number of fruits produced by the plant), and/or greater pollen tube length, in plants grown under conditions of elevated temperature stress, all in comparison to an untreated control. The skilled practitioner will recognize that in cotton, increased fruiting may be evidenced by an increased number of cotton bolls. The actual increase in tolerance exhibited by the resultant transgenic plants will vary with the particular heat shock protein and promoter used, as well as host plant and the variety thereof, soil conditions, and geography. As a practical matter, transgenic plants produced in accordance with this invention will exhibit an increase in mature pollen viability or increase in fruiting of at least about 15%, preferably about 20%, and most preferably about 25% or higher, all in comparison to a non-transformed control (measured at a confidence level of at least 80%, preferably measured at a confidence level of 95%).

One of skill in the art will recognize that, after the construct comprising the heat shock protein encoding sequence operatively linked to a pollen active promoter is stably incorporated in transgenic plants and confirmed to be operable, plant tissue or plant parts of the transgenic plants may be harvested, and/or the seed collected. The seed may serve as a source for growing additional plants with tissues or parts having the desired characteristics of producing pollen resistant to high temperatures. The construct may also be introduced into other plants by sexual crossing of the transformed plants. Any of a number of standard breeding techniques can be used, depending upon the species to be crossed.

The following examples are intended only to further illustrate the invention and are not intended to limit the scope of the invention which is defined by the claims.

### EXAMPLES

The Arabidopsis thaliana heat shock protein 101 was placed under the control of the constitutive ocs/mas ‘superpromoter’, incorporated into an expression vector and transferred into cotton hypocotyls and tobacco leaf disc cells via Agrobacterium. Enhanced heat tolerance of tobacco pollen from the transgenic plants has been identified via in vitro pollen germination studies. Both primary transformants and homozygous transgenic individuals from a segregating F3 population derived from a backcross with non-transgenic SR1 tobacco exhibited enhanced pollen germination and greater pollen tube lengths following a heat exposure. Increased boll set and greater seed numbers also were observed in transgenic cotton exposed to elevated day and night temperatures in greenhouse and field studies.

### Example 1

The binary vector pE1801-ocs/mas ‘superpromoter’-HSP101 was introduced into EHA 105 strain of Agrobacterium tumefacians (Hood et al., Transgenic Research, 2:208-218 (1993)) by direct transformation as described by Walker-Peach and Velten, in Plant Molecular Biology Manual, section B1:1-19 (Gelvin, Shilperoot and Verma, eds., Kluwer Academic Publishers, Dordrecht, The Netherlands, 1994)). The Agrobacterium was grown, with its proper selective antibiotics, in 5 ml, of LB. The newly grown bacterium was diluted 1:4 in a sterile tube containing LB broth. The solution was gently agitated until the bacteria became suspended in the LB. A turgid tobacco leaf was sterilized for 8 minutes in a 20% Sodium Hypo chlorite (generic bleach 5.25% by weight) and 0.1% SDS solution followed by treatment in 70% ethanol. Leaf punches were dropped into an MSIO (0.44% Murashige/Skoog basal salts, 3% Sucrose, 0.1 ug/ml naphtaleneacetic acid, and 1.0 ug/ml benzilaminopurine) petri plate. The contents of the inoculum were poured into the petri plate containing the explants. The explants were co-incubate with the bacterium for 24 hours at 28° C. with a 16/8 hour light cycle.

The leaf disks were transferred into a MS10 plate supplemented with Kanamycin (150 mg/L)+Carbenicillin (500 mg/L). Leaf disks were transferred onto fresh plates of MS10 Kanamycin (150 mg/L)+Carbenicillin (500 mg/L) at 2 week intervals. When callus began to grow, excess portions of the tumorous mass were removed. When the callus mass differentiated into a visible shoot with at least four well formed leaves and a 3 mm stem it was excised and transferred to rooting media. This media consists of the basic ingredients of the regeneration media but without BAP as the active hormone. Selection for the transformants was still maintained by Kanamycin at 150 mg/L and 350 mg/L Carbenicillin for the Agro strain. Once the regenerants had a well-developed root system, they were transferred to sterile soil and placed in an aquarium containing water plus a plant food additive with a clear top to allow humidity to accumulate to a high level.

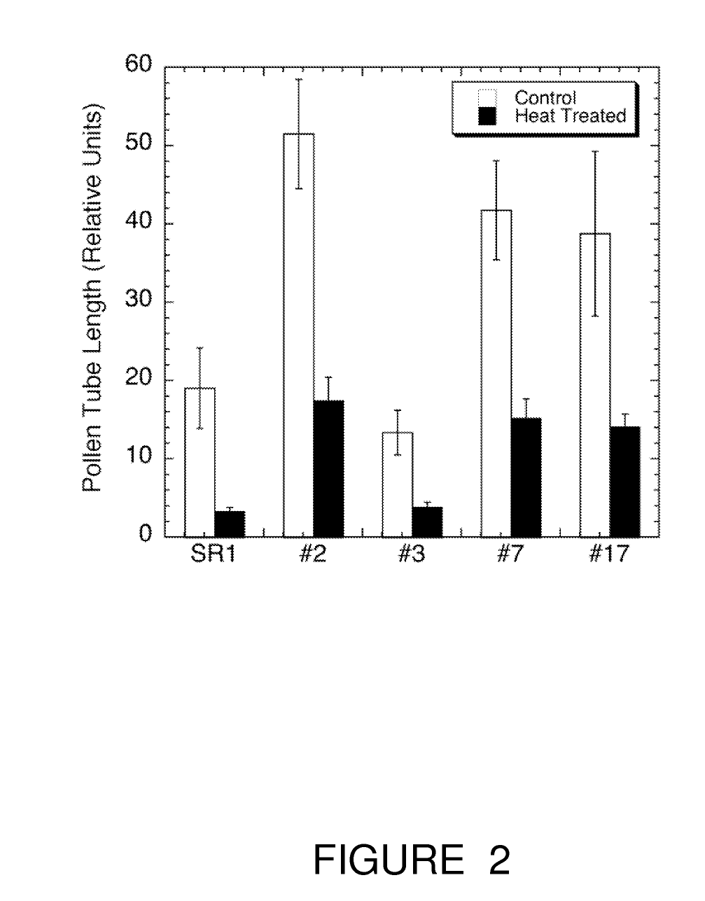

Twenty-four R0 plants were isolated and four were identified by antibody analyses as expressing high levels of HSP101. Selected plants were selfed and homozygous plants obtained for analysis of pollen heat tolerance. FIG. 2 is a graph of control (SR1) and HSP101 lines (#2, 3, 7, and 17)) tobacco pollen tube lengths before (Control) and after (Heat Treated) heat treatment. Longer pollen tube lengths were observed in three of the four transgenic lines (#2, 7, 17) compared to the SR1 pollen prior to heat treatment. The ratio of pollen tube lengths after heat treatment compared to pollen tube lengths prior to heat treatment were greater in all transgenic lines compared to the SR1 control. The greatest protection from heat injury was observed in lines #2, 7, and 17.

### Example 2

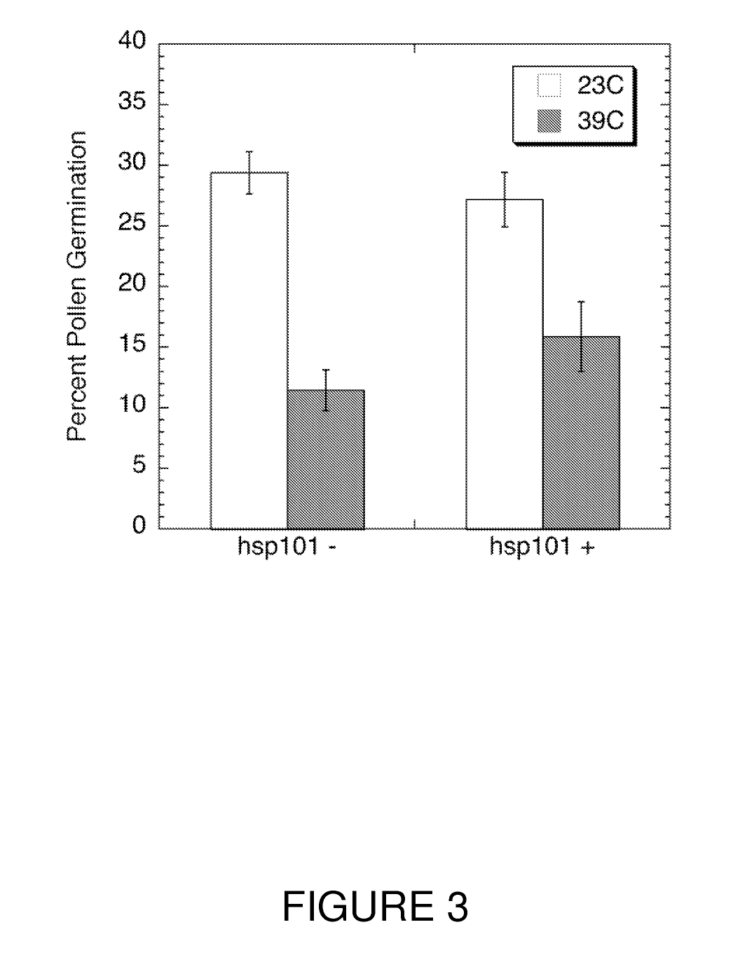

The binary vector pE1801-ocs/mas ‘superpromoter’-HSP101 was introduced into the EHA 105 strain of Agrobacterium tumefacians (Hood et al., 1983, Transgenic Research, 2:208-218) by direct transformation as described by Walker-Peach and Velten, in Plant Molecular Biology Manual, section B1:1-19 (Gelvin, Shilperoot and Verma, eds., Kluwer Academic Publishers, Dordrecht, The Netherlands, 1994). The constructs were subsequently introduced by Agrobacterium transfection into hypocotyl explants, by cutting submerged hypocotyls in a 24-hour-old culture of EHA 105, containing the appropriate construct, grown at 28° C. The hypocotyl sections were blotted dry on sterile filter paper to remove excess EHA 105, and transferred onto T2 Media (4.4 g/L MS medium with Gamborg vitamins+0.1 mg/L 2,4-D and 0.5 mg/L kinetin+30 g/L D-(+)-glucose+2 g/L phytagel). The infected hypocotyls tissue was incubated on T2 medium at 28° C. for 2 days prior to transfer to MS2NK CL medium (4.4 g/L MS medium with Gamborg vitamins+2 g/L phytagel+30 g/L D-(+)-glucose+2 mg/L alpha-naphthaleneacetic acid+0.1 mg/L kinetin+266 mg/L cefotaxime). Hypocotyls were transferred to fresh MS2NK CL medium three weeks following Agrobacterium infection. Four weeks after the transfer, calli were cut from the hypocotyls ends and moved onto MS2NK 1/4CL medium (4.4 g/L MS medium with Gamborg vitamins+2 g/L phytagel+30 g/L D-(+)-glucose+2 mg/L alpha-naphthaleneacetic acid+0.1 mg/L kinetin+67 mg/L cefotaxime). Six to seven weeks following the transfer to MS2NK 1/4CL medium the calli were moved into MSNH cell suspension medium (4.4 g/L MS medium with Gamborg vitamins+30 g/L D-(+)-glucose) and placed on a rotary shaker at 110 rpm. After 9 days on the shaker, cell suspensions were transferred to MSK medium (4.4 g/L MS medium with Gamborg vitamins+30 g/L D-(+)-glucose+1.9 g/L KNO₃+2 g/L phytagel). Immediately upon transfer of the embryogenic cell suspensions to MSK plates, the MSK plates with cell suspension were placed in a 50° C. incubator for 150 min. Petri dishes were stacked 5 plates high on each of 3 shelves within the incubator. Following the heat treatment, the Petri dishes were moved to a 28° C. tissue culture room and embryo development followed over a 9-day period. PCR-positive embryos were identified and plants were regenerated. Homozygous positive and negative plants were obtained for subsequent testing. Anti-hsp101 antibodies were used to evaluate hsp101 accumulation in pollen and leaves of the transgenic plants. The control plants showed hsp101 only in the leaves of the heat-treated plants. Cotton pollen was evaluated for heat sensitivity by germinating the pollen from greenhouse-grown cotton on a pollen germination media developed by Burke at either 23° C. or 39° C. for one hour. FIG. 3 shows the percent pollen germination of control (hsp101-) and transgenic (hsp101+). Improved heat tolerance was observed in the transgenic pollen compared with the control pollen.

### Example 3

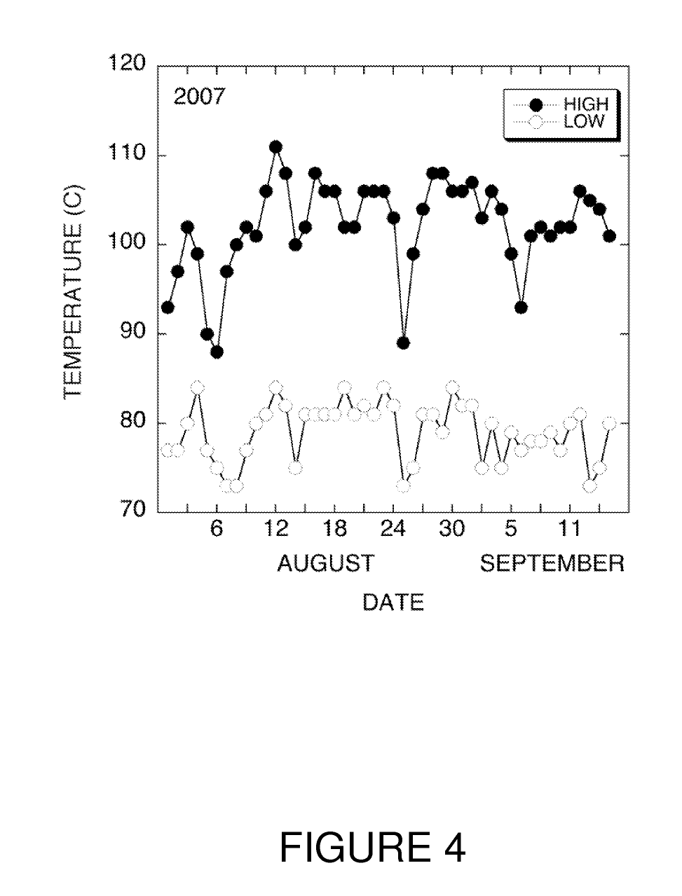

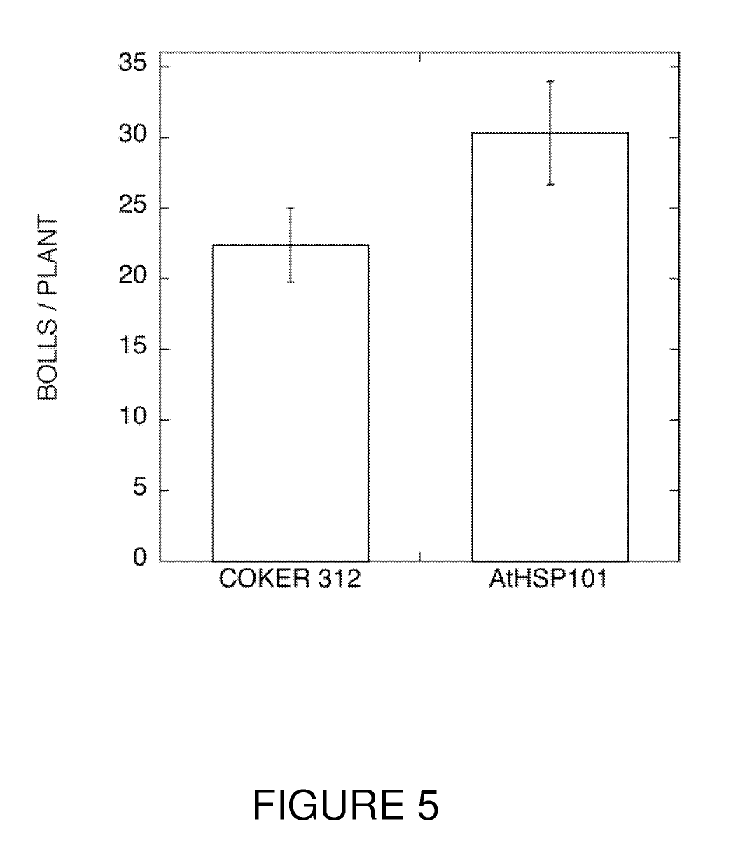

Homozygous positive and negative cotton plants obtained according the procedure described in Example 2 were evaluated for boll development in a field study performed in Maricopa, Ariz. High and low temperatures for the boll development period are shown in FIG. 4. Day time temperatures of over 100° F. were common throughout the reproductive period of growth. Bolls were harvested from 20 individual plants from hsp101+plants and hsp101-plants. FIG. 5 shows the yield enhancement of the hsp101+plants. A yield increase of 28% was observed for the hsp101+plants.

It is understood that the foregoing detailed description is given merely by way of illustration and that modifications and variations may be made therein without departing from the spirit and scope of the invention.

I claim:

1. A method for producing a transgenic plant comprising:
a) providing a plant, plant tissue or plant cell which is capable of regeneration,
b) transforming said plant, plant tissue or plant cell with a DNA construct comprising a nucleic acid coding sequence encoding a heat shock protein operatively linked to a promoter effective for expression in mature pollen of said plant, and
c) generating a transgenic plant from the transformed plant, plant tissue or plant cell.

2. The method of claim 1 further comprising selecting transgenic plants producing mature pollen which exhibits significantly increased tolerance to elevated temperature stress in comparison to an non-transformed control plant.

3. The method of claim 1 wherein said plant is selected from the group consisting of cotton, maize, wheat, soybeans, sorghum, oats, and barley.

4. The method of claim 3 wherein said plant is cotton.

5. The method of claim 1 wherein said heat shock protein is selected from the group consisting of a heat shock protein of the HSP 100 family and a heat shock protein of the HSP 70 family.

6. The method of claim 5 wherein said heat shock protein is a heat shock protein of the HSP 100 family.

7. The method of claim 1 wherein said plant which has not been transformed with said construct does not express said heat shock protein in said pollen which is mature.

8. A transgenic plant cell that comprises a DNA construct comprising a nucleic acid coding sequence encoding a heat shock protein operatively linked to a promoter effective for expression in mature pollen of said plant, plant tissue or plant cell.

9. The transgenic plant of claim 8 wherein said mature pollen exhibits significantly increased tolerance to elevated temperature stress in comparison to an non-transformed control plant.

10. The transgenic plant of claim 8 selected from the group consisting of cotton, maize, wheat, soybeans, sorghum, oats, and barley.

11. The transgenic plant of claim 10 comprising cotton.

12. The transgenic plant of claim 8 wherein said heat shock protein is selected from the group consisting of a heat shock protein of the HSP 100 family and a heat shock protein of the HSP 70 family.

13. The transgenic plant cell, plant tissue or plant of claim 12 wherein said heat shock protein is a heat shock protein of the HSP 100 family.

14. The transgenic plant of claim 8 wherein mature pollen of a plant cell, plant tissue or plant which has not been transformed with said construct does not express said heat shock protein.

15. A seed of the transgenic plant of claim 8.
